(19) United States
(12) Patent Application Publication
(10) Pub. No.: US 2025/0275745 A1
(43) Pub. Date: Sep. 4, 2025

(54) METHOD FOR QUANTIFYING BREAST DENSITY AND ULTRASONIC IMAGING SYSTEM

(71) Applicant: GE Precision Healthcare LLC, Waukesha, WI (US)

(72) Inventors: Liye Pei, Wuxi (CN); Ping Liu, Wuxi (CN); Xiaoyan Qin, Wuxi (CN); Bo Dan, Wuxi (CN)

(21) Appl. No.: 19/061,813

(22) Filed: Feb. 24, 2025

(30) Foreign Application Priority Data
Feb. 29, 2024 (CN) 202410231324.X

(51) Int. Cl. A61B 8/08; A61B 8/00; G06T 7/00; G06T 7/11

(52) U.S. Cl. CPC A61B 8/0825; A61B 8/44; A61B 8/5223; G06T 7/0012; G06T 7/11; G06T 2207/10132; G06T 2207/30068

(57) ABSTRACT

A method for quantifying breast density and an ultrasonic imaging system including performing ultrasonic scanning on a breast to acquire an ultrasonic image of the breast; determining a volume of a mammary gland and a volume of the breast based on the ultrasonic image; and determining the breast density based on the volume of the mammary gland and the volume of the breast. According to the embodiments of the present application, the breast density is quantified by acquiring three-dimensional volume data of the breast, and detecting a glandular breast tissue and a chest wall tissue from the three-dimensional volume data.

### CROSS-REFERENCE TO RELATED APPLICATION

This application claim priority to Chinese Patent Application No. 202410231324.X, which was file on Feb. 29, 2024 at the Chinese Patent Office. The entire contents of the above-listed application are incorporated by reference herein in their entirety.

### TECHNICAL FIELD

The present application relates to the field of medical imaging, and in particular to a method for quantifying breast density and an ultrasonic imaging system.

### BACKGROUND

Ultrasonic imaging is one of the important means for imaging the interior of the body of a person to be scanned. Generally, ultrasonic imaging systems use ultrasonic transducers to convert electrical energy into ultrasonic pulses. The ultrasonic pulses are sent to the interior of the body of the person to be scanned, and then echo signals are generated. The echo signals are received by a transducer element and then converted into electrical signals. The electrical signals are processed by a specialized processing device to then form a desired ultrasonic image.

Ultrasonic imaging systems have important applications in scanning many organs of the body. For example, a full-field breast ultrasonic scanning device may be used to image breast tissue in one or more planes.

It should be noted that the above introduction of the background is only for the convenience of clearly and completely describing the technical solutions of the present application, and for the convenience of understanding for those skilled in the art. The above technical solutions are not considered to be well known to those skilled in the art merely because they are set forth in the Background of the present application.

### SUMMARY

It has been found that breast density is a key factor in predicting cancer. Currently, quantification of the breast density is mostly implemented through mammography. However, such a method relies on the operator's technique and doctor's relevant clinical experience. There is a certain difficulty in achieving complete scanning for some larger, thicker, and more active mammary glands. Moreover, long-time exposure to an X-ray environment puts both the examinee and the doctor at risk.

To address at least one of the above problems or other similar problems, embodiments of the present application provide a method for quantifying breast density and an ultrasonic imaging system. Breast density is quantified by acquiring three-dimensional volume data of a breast, and then detecting a mammary gland tissue and a chest wall tissue from the three-dimensional volume data. Optionally, distribution of the breast density may also be visualized and displayed, providing more information for clinical applications, such as lesion detection navigation.

According to one aspect of the embodiments of the present application, a method for quantifying breast density is provided. The method includes:
performing ultrasonic scanning on a breast to acquire an ultrasonic image of the breast;
determining a volume of a mammary gland and a volume of the breast based on the ultrasonic image; and
determining the breast density based on the volume of the mammary gland and the volume of the breast.

In some embodiments, the performance of ultrasonic scanning on a breast to acquire an ultrasonic image of the breast includes:
using a scanning assembly to perform ultrasonic scanning on the breast, of which an outer edge is attached with at least one acoustically opaque marker, to acquire the ultrasonic image,
wherein the scanning assembly includes a frame, the frame accommodates a scanning probe and a driving device, and the driving device drives the scanning probe to move within the frame to perform the ultrasonic scanning.

In some embodiments, the marker includes a plurality of markers, and the plurality of markers are attached to the outer edge of the breast.

In some embodiments, the ultrasonic image includes a plurality of two-dimensional images.

In some embodiments, the determination of a volume of a mammary gland and a volume of the breast based on the ultrasonic image includes:
segmenting each of the plurality of two-dimensional images to acquire the mammary gland and a chest wall of the breast;
reconstructing each of the plurality of two-dimensional images to acquire a local coronal view of the breast; and
determining the volume of the mammary gland based on the mammary gland, and determining the volume of the breast based on the chest wall and the local coronal views.

In some embodiments, the determination of the volume of the mammary gland based on the mammary gland includes:
segmenting the mammary gland from each of the plurality of two-dimensional images to acquire a segmentation mask of the mammary gland;
registering the plurality of segmentation masks of the mammary gland on the plurality of two-dimensional images based on a registration parameter used for registering the plurality of local coronal views of the breast to acquire a mask of the mammary gland; and
acquiring the volume of the mammary gland based on the mask of the mammary gland.

In some embodiments, the determination of the volume of the breast based on the chest wall and the local coronal views includes:
detecting a nipple and markers on each of the plurality of local coronal views;
registering the plurality of local coronal views based on positions of the nipple and the markers on each of the local coronal views to acquire an overall coronal view of the breast;
determining a breast boundary based on the positions of the markers; and
acquiring the volume of the breast based on the breast boundary and a distance from the skin to the chest wall within the breast boundary.

In some embodiments, the determination of the volume of the breast based on the chest wall and the plurality of local coronal views further includes:
determining a position of the chest wall.

In some embodiments, the determination of a position of the chest wall includes:
segmenting the chest wall from each of the plurality of two-dimensional images to acquire a segmentation mask of the chest wall;
registering the plurality of segmentation masks of the chest wall on the plurality of two-dimensional images based on a registration parameter used for registering the plurality of local coronal views of the breast to acquire a mask of the chest wall; and
acquiring a position of the chest wall based on the mask of the chest wall.

In some embodiments, the method further includes:
calculating a breast density map within an area of the breast based on a thickness of the mammary gland and a distance from the skin to a chest wall; and
visualizing and displaying the breast density map.

In some embodiments, the method further includes:
overlaying and displaying the breast density map on a coronal view of the breast.

In some embodiments, the method further includes:
determining the thickness of the mammary gland and a position of the chest wall based on the ultrasonic image.

In some embodiments, the determination of the thickness of the mammary gland based on the ultrasonic image includes:
segmenting the mammary gland from each two-dimensional image to acquire a segmentation mask of the mammary gland;
registering the plurality of segmentation masks of the mammary gland on the plurality of two-dimensional images based on a registration parameter used for registering a plurality of local coronal views of the breast to acquire a mask of the mammary gland; and
acquiring the thickness of the mammary gland from calculation based on the mask of the mammary gland.

In some embodiments, the determination of a position of the chest wall based on the ultrasonic image includes:
segmenting the chest wall from each two-dimensional image to acquire a segmentation mask of the chest wall;
registering the plurality of segmentation masks on the plurality of two-dimensional images based on a registration parameter used for registering a plurality of local coronal views of the breast to acquire a mask of the chest wall; and
acquiring a position of the chest wall based on the mask of the chest wall.

According to another aspect of the embodiments of the present application, an ultrasonic imaging system is provided. The system includes:
a scanning assembly moving over the surface of a breast to acquire ultrasonic echo signals; and
a processor configured to perform the method according to any one of the foregoing embodiments.

According to still another aspect of the embodiments of the present application, provided is a non-transitory computer-readable medium, having a computer program stored thereon, where the computer program has at least one code segment, and the at least one code segment is executable by a machine so that the machine performs steps of the method according to any one of the foregoing embodiments.

A beneficial effect of the embodiments of the present application is that, according to the method in the embodiments of the present application, breast density is quantified by acquiring three-dimensional volume data of a breast, and detecting a glandular breast tissue and a chest wall tissue from the three-dimensional volume data. Optionally, distribution of the breast density may also be visualized and displayed, providing more information for clinical applications, such as lesion detection navigation.

With reference to the following description and drawings, specific implementations of the present application are disclosed in detail. It should be understood that the implementations of the present application are not limited in scope thereby. Within the scope of the spirit and clauses of the appended claims, the embodiments of the present application include many changes, modifications, and equivalents.

The features described and/or illustrated for one implementation may be used in one or more other implementations in the same or similar manner, be combined with features in other embodiments, or replace features in other implementations.

It should be emphasized that the term “include/comprise/have”, when used herein, refers to the presence of features, integrated components, or assemblies, but does not preclude the presence or addition of one or more other features, integrated components, or assemblies.

### BRIEF DESCRIPTION OF THE DRAWINGS

The above and other objects, features, and advantages of the embodiments of the present application will become more apparent from the following detailed description with reference to the drawings, in which:

FIG. 1 is a perspective view of an ultrasonic imaging system according to an embodiment of the present application;

FIG. 2 is a block diagram of the ultrasonic imaging system according to the embodiment of the present application;

FIG. 3 is a perspective view of a scanning assembly of the ultrasonic imaging device according to the embodiment of the present application;

FIG. 4 is a schematic diagram of a method for quantifying breast density according to an embodiment of the present application;

FIG. 5 is a schematic diagram of an example of a breast attached with markers M;

FIG. 6 is a schematic diagram of determining a volume of a mammary gland and a volume of the breast based on an ultrasonic image;

FIG. 7 is a schematic diagram of segmenting a mammary gland and a chest wall from a two-dimensional image;

FIG. 8 is a schematic diagram of a local coronal view of a breast;

FIG. 9 is a schematic diagram of determining a volume of a breast based on a local coronal view of a chest wall and the breast;

FIG. 10 is a schematic diagram of an overall coronal view of a breast;

FIG. 11 is a schematic diagram of determining a position of a chest wall;

FIG. 12 is a schematic diagram of determining a volume of a mammary gland based on the mammary gland;

FIG. 13 is a schematic diagram of calculation of a breast density map;

FIG. 14 is a schematic diagram of an example of a breast density map; and

FIG. 15 is a schematic diagram of an example of overlaying and displaying the breast density map shown in FIG. 14 on a coronal view of the breast.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the present application may be employed are indicated. It should be understood that the present application is not limited to the described implementations, and include all modifications, variations, and equivalents which fall within the scope of the appended claims.

In the embodiments of the present application, the terms “first”, “second”, “upper”, “lower”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part based on . . . ”, unless otherwise specified in the context.

The features described and/or illustrated for one implementation may be used in one or more other implementations in the same or similar way, be combined with features in other embodiments, or replace features in other implementations.

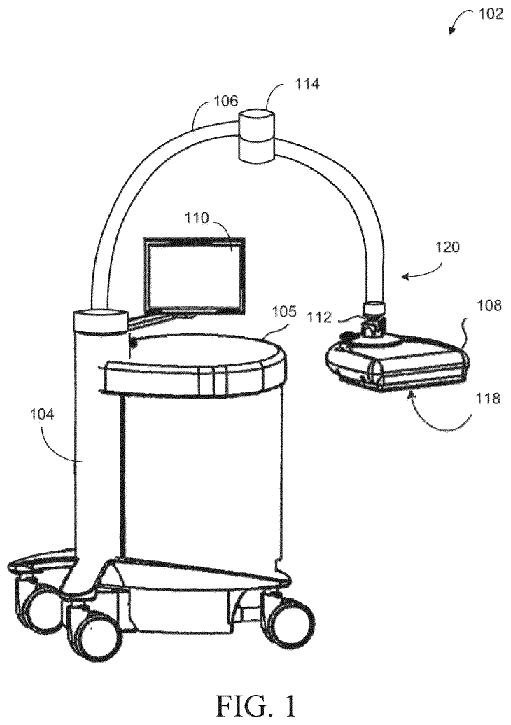

FIG. 1 shows a perspective view of an ultrasonic imaging device 102 according to some embodiments. As shown in FIG. 1, a body of the ultrasonic imaging system 102 may include a main device, a display 110, an adjustable arm 106, and a scanning assembly 108. The main device may include a body frame 104, an ultrasonic processor housing 105, and an ultrasonic processor inside the housing 105. The specific structure of each component will be illustrated in detail below.

The body frame 104, the ultrasonic processor housing 105 containing the ultrasonic processor, a movable and adjustable support arm (for example, an adjustable arm) 106 including a hinge joint 114, the scanning assembly 108 connected to a first end 120 of the adjustable arm 106 by means of a ball and socket connector (for example, a ball joint) 112, and the display 110 connected to the body frame 104. The display 110 is connected to the body frame 104 at a joining point where the adjustable arm 106 enters the body frame 104. Since the display 110 is directly connected to the body frame 104 rather than the adjustable arm 106, the display 110 does not affect the weight of the adjustable arm 106 and a balancing mechanism of the adjustable arm 106. In one example, the display 110 is rotatable in horizontal and transverse directions (for example, rotatable around a central axis of the body frame 104), but is not vertically movable. In an alternative example, the display 110 may also be vertically movable. Although FIG. 1 depicts the display 110 connected to the body frame 104, in other examples, the display 110 may be connected to different components of the imaging system 102, such as connected to the ultrasonic processor housing 105, or positioned away from the imaging system 102.

In one embodiment, the adjustable arm 106 is configured and adapted such that the pressing/scanning assembly 108 (i) is neutrally buoyant in space, or (ii) has a light net downward weight (for example, 1-2 kg) for pressing the breast, while allowing easy user operation. In an alternative embodiment, the adjustable arm 106 is configured such that the scanning assembly 108 is neutrally buoyant in space during positioning of a scanner on tissue of a patient. Then, after the scanning assembly 108 is positioned, internal components of the imaging system 102 may be adjusted to apply a desired downward weight for pressing the breast and improving image quality. In one example, the downward weight (for example, a force) may be in the range of 2-11 kg.

As described above, the adjustable arm 106 includes the hinge joint 114. The hinge joint 114 divides the adjustable arm 106 into a first arm portion and a second arm portion. The first arm portion is connected to the scanning assembly 108 and the second arm portion is connected to the body frame 104. The hinge joint 114 allows the second arm portion to rotate relative to the second arm portion and the body frame 104. For example, the hinge joint 114 allows the scanning assembly 108 to translate transversely and horizontally, but not vertically, relative to the second arm portion and the body frame 104. In such manner, the scanning assembly 108 can rotate toward the body frame 104 or away from the body frame 104. However, the hinge joint 114 is configured to allow the entire adjustable arm 106 (for example, the first arm portion and the second arm portion) to move vertically together as a whole (for example, translating upward and downward along with the body frame 104).

The scanning assembly 108 may include a film assembly 118 having a film that is in a substantially tensioned state to be at least partially attached, for pressing the breast. The film assembly 118 has a bottom surface for contacting the breast, and when the bottom surface is in contact with the breast, the transducer sweeps over a top surface of the film to scan the breast. In one example, the film is a tensioned fabric sheet.

The film assembly 118 may further include an outer frame and a film. The film is fixedly disposed in the outer frame, and the outer frame is detachably connected to the scanning assembly. In an ultrasonic imaging process performed by the ultrasonic imaging system, one side surface of the film can be at least partially in contact with an ultrasonic transducer, and another side surface of the film is at least partially in contact with a tissue to be scanned. Such an arrangement can ensure that the ultrasonic transducer transmits and receives signals with less attenuation, and can fix the breast to be scanned to facilitate scanning.

Optionally, the adjustable arm may include a potentiometer (not shown) to allow position and direction sensing performed by the pressing/scanning assembly 108, or may use other types of position and direction sensing (such as gyroscope, magnetic, optical, and radio frequency (RF)). A fully functional ultrasonic engine may be provided within the ultrasonic processor housing 105, and is configured to drive the ultrasonic transducer, and generate volumetric breast ultrasound data from a scan in conjunction with related position and orientation information. In some examples, volumetric scan data may be transmitted to another computer system by using any of a variety of data transmission methods known in the art so as to be further processed, or the volumetric scan data may be processed by the ultrasonic engine. A general-purpose computer/processor integrated with the ultrasonic engine may further be provided for general user interface and system control. The general-purpose computer may be a self-contained stand-alone unit, or may be remotely controlled, configured, and/or monitored by remote stations connected across networks.

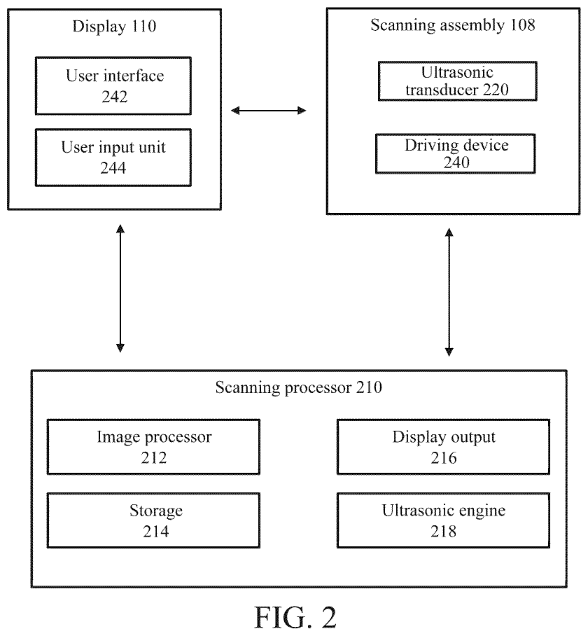

FIG. 2 is a block diagram 200 that schematically illustrates various system parts of the ultrasonic imaging system 102. As shown in FIG. 2, the ultrasonic imaging system 102 includes a scanning assembly 108, a display 110, and a scanning processor 210. In one example, the scanning processor 210 may be included within the ultrasonic processor housing 105 of the imaging system 102. As shown in FIG. 2, the scanning assembly 108, the display 110, and the scanning processor 210 are separate components in communication with each other. However, in some embodiments, one or more of these components may be integrated (for example, the display and the scanning processor may be included in a single component).

In the example of FIG. 2, the scanning assembly 108 includes at least an ultrasonic transducer 220 and a driving device 240. The ultrasonic transducer 220 includes a transducer array of transducer elements, such as a piezoelectric element converting electrical energy into ultrasonic waves and then detecting reflected ultrasonic waves.

The scanning assembly 108 may communicate with the scanning processor 210 to send raw scan data to an image processor. The scanning assembly 108 may optionally communicate with the display 110 so as to indicate a user to reposition the scanning assembly as described above, or to receive information from the user (via user input 244).

In the example of FIG. 2, the scanning processor 210 includes an image processor 212, a memory 214, display output 216, and an ultrasonic engine 218. The ultrasonic engine 218 may drive activation of the transducer elements of the transducer 220, and in some embodiments, the driving device 240 may be activated. Furthermore, the ultrasonic engine 218 may receive raw image data (for example, ultrasonic echoes) from the scanning assembly 108. The raw image data may be sent to the image processor 212 and/or a remote processor (for example, via a network) and be processed to form a displayable image of a tissue sample. It should be understood that in some embodiments, the image processor 212 may be included in the ultrasonic engine 218.

In the example shown in FIG. 2, information may be transmitted from the ultrasonic engine 218 and/or the image processor 212 to the user of the imaging system 102 via a display output 216 of the scanning processor 210. In one example, the user of the ultrasonic imaging system may include an ultrasonic technician, a nurse, or a physician such as a radiologist. For example, a processed image of scanned tissue may be sent to the display 110 via the display output 216. In another example, information (such as the progress of scanning) related to parameters of the scanning may be sent to the display 110 via the display output 216. The display 110 may include a user interface 242 configured to display images or other information to the user. Furthermore, the user interface 242 may be configured to receive an input from the user (such as by means of a user input unit 244), and send the input to the scanning processor 210. In one example, the user input unit 244 may be a touch screen of the display 110. However, other types of user input mechanisms are also possible, such as a mouse, a keyboard, and the like.

The scanning processor 210 may further include the memory 214. The memory 214 may include movable and/or permanent devices, and may include an optical memory, a semiconductor memory, and/or a magnetic memory. The memory 214 may include a volatile, non-volatile, dynamic, static, read/write, read only, random access, sequential access, and/or annex memory. The memory 214 may store non-transitory instructions executable by a controller or processor (such as a controller 218 or the image processor 212) so as to perform one or more methods or routines as described below. The memory 214 may store raw image data received from the scanning assembly 108, processed image data received from the image processor 212 or the remote processor, and/or additional information.

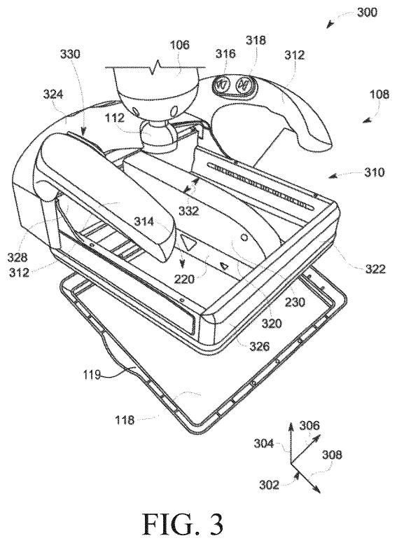

FIG. 3 shows a schematic diagram 300 of an isometric view of the scanning assembly 108 connected to the adjustable arm 106. As shown in FIG. 3, the schematic diagram 300 includes a coordinate system 302, and the coordinate system 302 includes a vertical axis 304, a horizontal axis 306, and an abscissa axis 308.

The scanning assembly 108 includes a housing 310, the transducer module 220, and the module receiver 230. The housing 310 includes a frame 322 and a handle portion 324, and the handle portion includes two handles 312. The two handles 312 oppose each other across a transverse axis of the scanning assembly 108, and the transverse axis is centered on the adjustable arm 106 and defined relative to the transverse axis 308. The frame 322 is rectangular, and an inner periphery of the frame 322 defines an opening 314. The opening 314 provides space (e.g., a void volume) for translating the module receiver 230 and the transducer module 220 during a scanning process. In another example, the frame 322 can have another shape, such as a square having the square opening 314. In addition, the frame 322 has a thickness defined between an inner periphery and an outer periphery of the frame 322.

The frame 322 includes four sets of side walls (e.g., a set including inner and outer side walls, the inner side walls defining the opening 314). In particular, the frame 322 includes a front side wall 326 and a rear side wall 328, the rear side wall 328 is directly connected to the handle portion 324 of the housing 310, and the front side wall 326 is opposite to the rear side wall 328 with respect to the horizontal axis 306. The frame 322 further includes right and left side walls, the corresponding side walls opposing each other and both being in a plane defined by the vertical axis 304 and the transverse axis 308.

The frame 322 of the housing 310 further includes a top side and a bottom side, and the top side and the bottom side are defined relative to the vertical axis 304. The top side faces the adjustable arm 106. The film 118 is disposed across the opening 314. More specifically, the film 118 is connected to the bottom side of the frame 322. In an example, the film 118 is a diaphragm that remains tensioned across the opening 314. The film 118 may be made from a flexible but non-stretchable material, and the material is thin, waterproof, durable, highly acoustically transparent, resistant to chemical corrosion, and/or biocompatible. As described above, the bottom surface of the film 118 may contact a tissue (e.g., a breast) during scanning, and the upper surface of the film 118 may at least partially contact the transducer module 220 during scanning. As shown in FIG. 3, the film 118 is permanently connected to a hard-housing holding portion 119 surrounding the periphery of the film 118. The holding portion 119 is connected to the bottom side of the frame 322. In one example, the holding portion 119 can be fastened to a lip-like edge on the bottom side of the frame 322 of the housing 310, so that the film 118 does not become unconnected during scanning, but is still removably connected to the frame 322. The film 118 may not be permanently connected to the hard-housing holding portion 119, and thus the film 118 may be connected to the frame 322 without the hard-housing holding portion 119. Instead, the film 118 may be directly and removably connected to the frame 322.

The handle portion 324 of the housing 310 includes the two handles 312 for moving the scanning assembly 108 in space and positioning the scanning assembly 108 on a tissue (e.g., on the body of a patient). In an alternative embodiment, the housing 310 may not include the handle 312. In an example, the handle 312 may be integrally formed with the frame 322 of the housing 310. In another example, the handle 312 and the frame 322 may be formed separately and then mechanically connected together to form the entire housing 310 of the scanning assembly 108.

As shown in FIG. 3, the scanning assembly 108 is connected to the adjustable arm 106 by means of a ball joint 112 (e.g., a ball and socket connector). Specifically, a top dome portion of the handle portion 324 is connected to the ball joint 112. The top of the handle portion 324 includes a depression forming a socket, and a ball of the ball joint 112 is fit in the socket. The ball joint 112 is movable in multiple directions. For example, the ball joint 112 provides rotational motion of the scanning assembly relative to the adjustable arm 106. The ball joint 112 includes a locking mechanism for locking the ball joint 112 in place, thereby holding the scanning assembly 108 stationary relative to the adjustable arm 106. Furthermore, the ball joint 112 may also be configured to only rotate but not to move in multiple directions, such as oscillating.

Additionally, as shown in FIG. 3, the handle 312 of the handle portion 324 includes buttons for controlling scanning and adjusting the scanning assembly 108. Specifically, a first handle of the handles 312 includes a first weight adjustment button 316 and a second weight adjustment button 318. The first weight adjustment button 316 may reduce a load applied to the scanning assembly 108 from the adjustable arm 106. The second weight adjustment button 318 may increase a load applied to the scanning assembly 108 from the adjustable arm 106. Increasing the load applied to the scanning assembly 108 may increase the pressure and the amount of pressing applied to the tissue on which the scanning assembly 108 is placed. Furthermore, increasing the load applied to the scanning assembly increases the effective weight of the scanning assembly on the tissue to be scanned. In one example, increasing the load may press a tissue of a patient, such as a breast. In such a way, varying amounts of pressure (e.g., load) may be applied consistently with the scanning assembly 108 during scanning, so as to acquire high quality images by using the transducer module 220.

Before the scanning process, a user (e.g., an ultrasonic technician or physician) may position the scanning assembly 108 on a patient or a tissue. Once the scanning assembly 108 is properly positioned, the user may adjust a weight (e.g., adjusting an amount of pressing) of the scanning assembly 108 on the patient by using the first weight adjustment button 316 and/or the second weight adjustment button 318. Then, the user may initiate the scanning process by means of additional control on the handle portion 324 of the housing 310. For example, as shown in FIG. 3, the second handle of the handles 312 includes two additional buttons 330 (not separately shown). The two additional buttons 330 may include a first button for initiating a scan (e.g., once the scanning assembly has been placed on the tissue/patient and an amount of pressing has been selected) and a second button for stopping the scan. In one example, once the first button is selected, the ball joint 112 may be locked, thereby stopping transverse and horizontal movement of the scanning assembly 108.

The module receiver 230 is positioned within the housing 310. Specifically, the module receiver 230 is mechanically connected to a first end of the housing 310 at a rear side wall 328 of the frame 322, and the first end is closer to the adjustable arm 106 than a second end of the housing 310. The second end of the housing 310 is located at a front side wall 326 of the frame 322. In one example, the module receiver 230 is connected to the first end by means of a protruding portion of the module receiver 230, the protruding portion is connected to the motor, and the protruding portion is connected to the motor of the module receiver 230.

As described above, the housing 310 is configured to remain stationary during scanning. In other words, once the weight applied to the scanning assembly 108 is adjusted by means of the adjustable arm 106 and then the ball joint 112 is locked, the housing 310 may remain in the resting position without translating in the horizontal or transverse direction. However, the housing 310 may still translate vertically as the adjustable arm 106 moves vertically.

Instead, the module receiver 230 is configured to translate relative to the housing 310 during scanning. As shown in FIG. 3, the module receiver 230 translates horizontally along a horizontal axis 306 relative to the housing 310. The motor of the module receiver 230 may slide the module receiver 230 along an upper surface of the first end of the housing 310.

The transducer module 220 is removably connected to the module receiver 230. Therefore, during scanning, the transducer module 220 and the module receiver 230 translate horizontally. During scanning, the transducer module 220 sweeps horizontally across the breast under the control of the motor of the module receiver 230, and at the same time, a contact surface of the transducer module 220 contacts the film 118. The transducer module 220 and the module receiver 230 are connected together at a module interface 320. The module receiver 230 has a width 332 that is the same as a width of the transducer module 220. In an alternative embodiment, the width 332 of the module receiver may be different from the width of the transducer module 220. In some embodiments, the module interface 320 includes a connector between the transducer module 220 and the module receiver 230, and the connector includes mechanical and electrical connections.

In the examples in FIG. 1 to FIG. 3, the ultrasonic imaging system 102 may perform ultrasonic scanning using an automated breast ultrasonic imaging (ABUS) technology, and the ultrasonic imaging system 102 may also be referred to as an ABUS system. ABUS is a new high-resolution breast 3D ultrasonic imaging technology that can acquire imaging of an entire breast anatomy structure (including cross-sectional, sagittal, and coronal planes), allowing for observation of an internal structure of the breast from a 3D perspective and in multiple layers. For ease of description, the ABUS system is used as an example below, but the present application is not limited thereto. Other types of ultrasonic imaging systems that fulfill any of the embodiments described below are also allowed.

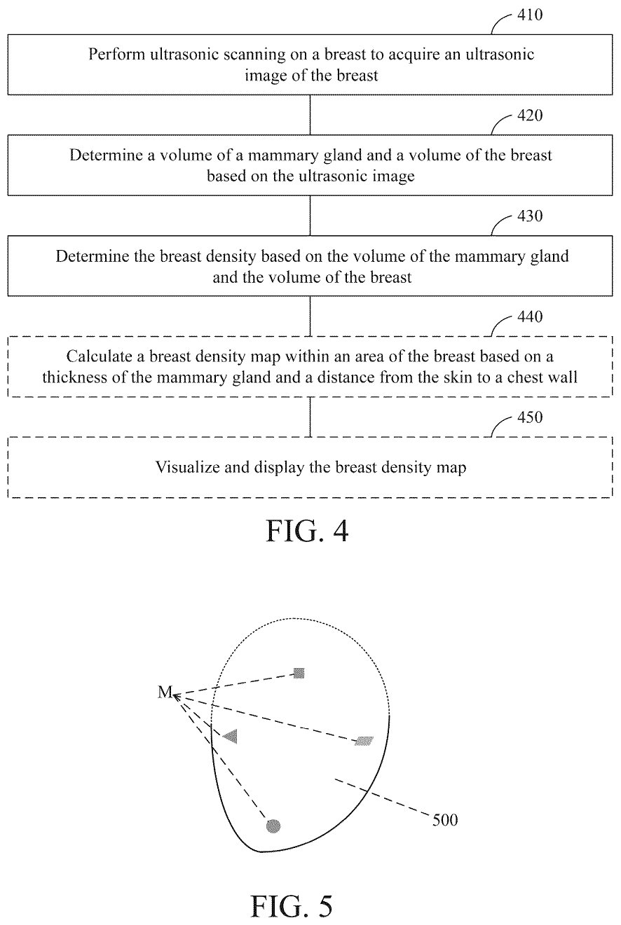

The embodiments of the present application provide a method for quantifying breast density. FIG. 4 is a schematic diagram of a method for quantifying breast density according to an embodiment of the present application. As shown in FIG. 4, the method includes:
410: performing ultrasonic scanning on a breast to acquire an ultrasonic image of the breast;
420: determining a volume of a mammary gland and a volume of the breast based on the ultrasonic image; and
430: determining the breast density based on the volume of the mammary gland and the volume of the breast.

It should be noted that FIG. 4 merely schematically illustrates the method for quantifying breast density according to an embodiment of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 4.

According to the above embodiments, breast density is quantified by acquiring three-dimensional volume data of a breast, and detecting a glandular breast tissue and a chest wall tissue from the three-dimensional volume data, so that accurately quantified breast density data is acquired while no potential radiation risk is incurred. Especially, compared with a qualitative approach to breast density evaluation, in the above embodiments of the present application, the volume of the breast and the volume of the mammary gland are qualitatively acquired, so that the breast density can be accurately determined. The above processes for acquiring the volumes are implemented through ultrasonic imaging, further ensuring the safety of evaluation.

In operation 410, the scanning assembly may be used to perform ultrasonic scanning on the breast, of which an outer edge is attached with at least one acoustically opaque marker, to acquire the ultrasonic image.

In the above embodiment, the scanning assembly is, for example, the scanning assembly 108 as shown in FIG. 1 to FIG. 3, and includes a frame 322. The frame 322 accommodates a scanning probe (a transducer module 220 and a module receiver 230) and a driving device 240. The driving device 240 drives the scanning probe to move within the frame 322 to perform the ultrasonic scanning.

In the above embodiment, the marker is made of an acoustically opaque material, such as a sticker, plastic, etc., and hence can generate a shadow on a coronal view of the ultrasonic image. Since the marker is attached to the outer edge of the breast, the shadow of the marker does not affect image quality of a breast area with a diagnostic value.

FIG. 5 is a schematic diagram of an example of a breast attached with markers M. As shown in FIG. 5, the markers M are respectively attached at four positions of the outer edge of the breast 500, for example, in directions of three o'clock, six o'clock, nine o'clock, and twelve o'clock of the breast 500. A boundary of the breast is outlined by the four markers M. In the embodiment of the present application, an ultrasonic image is acquired by performing ultrasonic scanning on the breast 500 attached with the markers M.

In the example shown in FIG. 5, for example, four markers M with different shapes are attached to the outer edge of the breast 500. The present application does not limit the number and shape of the markers M. For example, alternatively, other numbers of markers may be attached to the outer edge of the breast 500, markers with the same shape may be attached to the outer edge of the breast 500, or the like. For example, one, two, three, or more than four markers of the same shape or different shapes are attached to the outer edge of the breast 500.

In the embodiment of the present application, the outer edge of the breast may be understood in the art as an outer contour of the breast, which includes an entire mammary gland area and other body tissues, and which in the example in FIG. 5 is a range enclosed by the markers M. The specific positions for adhering the markers M are not limited in the present application. For example, in the example of FIG. 5, the four markers M are approximately equally spaced. Alternatively, the positions of the markers M may be non-equally spaced.

In some embodiments, the ultrasonic image includes a plurality of two-dimensional images, and a coronal view of the breast corresponding to the ultrasonic image can be acquired by reconstructing the plurality of two-dimensional images.

In the above embodiment, a plurality of two-dimensional images are acquired at each time of ultrasonic scanning, and the plurality of two-dimensional images are reconstructed to acquire a coronal view as a scanning result of the ultrasonic scanning at that time. After reconstruction, a three-dimensional ultrasonic image can be acquired. For example, ultrasonic scanning is performed on a triangular marker and a circular marker shown in FIG. 5 to acquire a plurality of two-dimensional images (in a direction perpendicular to the paper), and the plurality of two-dimensional images are reconstructed to acquire a local coronal view of the breast at the scanning position. Similarly, ultrasonic scanning is performed on the triangular marker and a square marker shown in FIG. 5 to acquire a coronal view of the breast at the scanning position. Ultrasonic scanning is performed on a square marker and a prismatic marker shown in FIG. 5 to acquire a local coronal view of the breast at the scanning position, and ultrasonic scanning is performed on the prismatic marker and the circular marker shown in FIG. 5 to acquire a local coronal view of the breast at the scanning position. Thus, four local coronal views are acquired by performing scanning on the breast four times, and an overall coronal view of the breast can be acquired from the four local coronal views. For example, the overall coronal view of the breast is acquired by splicing four local coronal views. The specific splicing method will be described later. It is not difficult to understand that the two-dimensional images can ensure a high resolution of breast scanning and integrity of a field of view as much as possible. In such a manner, higher image quality is provided, especially compared with conventional three-dimensional and four-dimensional volumetric ultrasonic imaging. Specifically, in the above manner, a three-dimensional image is acquired on the basis of acquired high-quality two-dimensional images, ensuring the high resolution of the three-dimensional image (volumetric image). This helps to identify the edge and the mammary gland of the breast from the two-dimensional images and reconstructed three-dimensional image, thus ensuring the accuracy of quantitative analysis.

The above is just an example. If the entire breast area can be covered by simply performing one ultrasonic scanning on the breast, the overall coronal view of the breast is acquired by one ultrasonic scanning. For another example, when the entire breast area can be covered by performing ultrasonic scanning on the breast three times, three local coronal views of the breast are acquired by the three times of ultrasonic scanning, and the overall coronal view of the breast is acquired by splicing the three local coronal views.

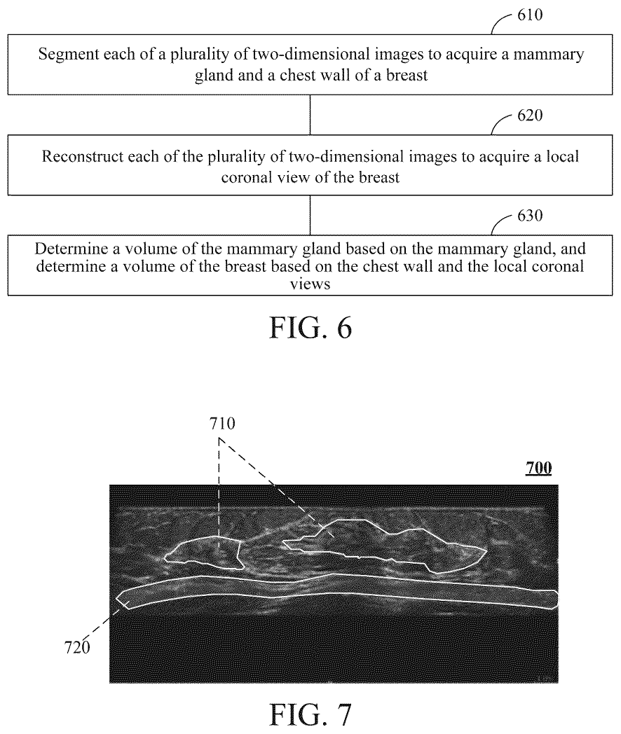

In operation 420, the determination of a volume of a mammary gland and a volume of the breast based on the ultrasonic image may be implemented, for example, in the method shown in FIG. 6. As shown in FIG. 6, the method includes:
610: segmenting each of a plurality of two-dimensional images to acquire a mammary gland and a chest wall of the breast;
620: reconstructing each of the plurality of two-dimensional images to acquire a local coronal view of the breast; and
630: determining a volume of the mammary gland based on the mammary gland, and determining a volume of the breast based on the chest wall and the local coronal views.

It should be noted that FIG. 6 merely schematically illustrates the method for determining the volume of the mammary gland and the volume of the breast based on the ultrasonic image according to an embodiment of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 6.

The above embodiment follows the example of performing scanning four times in FIG. 5. Accordingly, in operation 610, each of a plurality of two-dimensional images acquired at each time of ultrasonic scanning is segmented to acquire a mammary gland and a chest wall of the breast; in operation 620, each of the plurality of two-dimensional images acquired at each time of ultrasonic scanning is reconstructed to acquire a local coronal view of the breast; and in operation 630, a volume of the mammary gland is determined based on the mammary gland acquired by multiple times of ultrasonic scanning, and a volume of the breast is determined based on the chest wall and the local coronal views acquired by the multiple times of ultrasonic scanning.

In operation 610, regions of interest, such as the mammary gland and the chest wall, may be segmented from the two-dimensional images using an AI segmentation model. FIG. 7 is a schematic diagram of segmenting a mammary gland 710 and a chest wall 720 from a two-dimensional image 700. The specific segmentation method is not limited in the present application, for which reference may be made to the related technology.

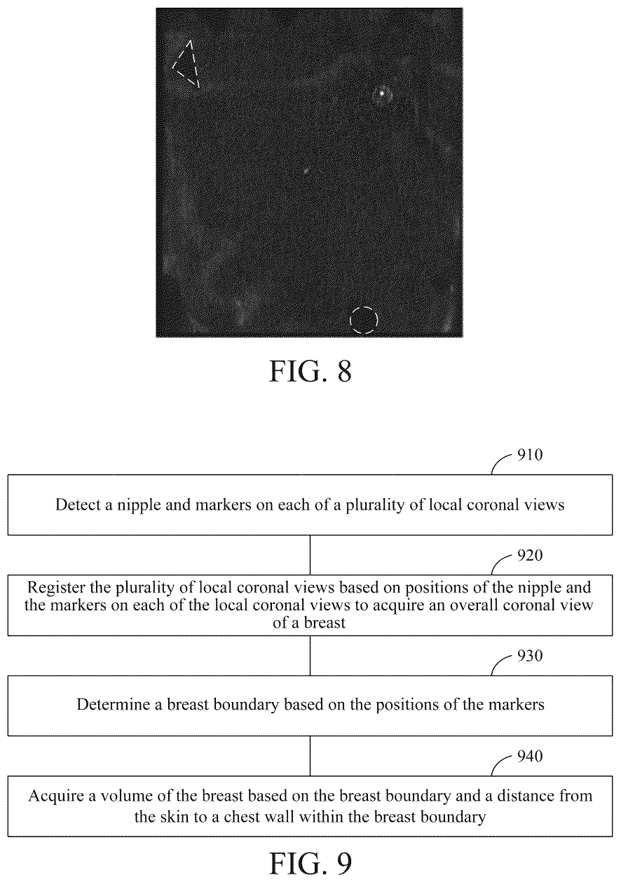

In operation 620, the two-dimensional images are reconstructed to acquire a local coronal view of the breast. FIG. 8 is a schematic diagram of a local coronal view of a breast. In the example of FIG. 8, for example, ultrasonic scanning is performed on the triangular marker and the circular marker shown in FIG. 5 to acquire a local coronal view. As shown in FIG. 8, since markers are attached to the outer edge of the breast, shadows of the markers are retained on the acquired local coronal view. Since the markers are attached to the outer edge of the breast, the shadows do not affect the image quality of the local coronal view.

In operation 630, the determination of a volume of the breast based on the local coronal views of the chest wall and the breast may be implemented, for example, in the method shown in FIG. 9. As shown in FIG. 9, the method includes:
910: detecting a nipple and markers on each of a plurality of local coronal views;
920: registering the plurality of local coronal views based on positions of the nipple and the markers on each of the local coronal views to acquire an overall coronal view of a breast;
930: determining a breast boundary based on the positions of the markers; and
940: acquiring a volume of the breast based on the breast boundary and a distance from the skin to a chest wall within the breast boundary.

It should be noted that FIG. 9 merely schematically illustrates the method for determining the volume of the breast based on the local coronal views of the chest wall and the breast according to an embodiment of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 9.

In operation 910, each local coronal view is acquired by performing ultrasonic scanning on a part of the breast. For example, ultrasonic scanning is performed on an area of the breast containing the triangular marker and the circular marker to acquire a plurality of two-dimensional images, and the plurality of two-dimensional images are reconstructed to acquire a local coronal view of the breast. A nipple and markers can be detected on the local coronal view. The present application does not specifically limit the detection method. For example, detection may be performed based on the brightness of a pixel on the image.

In the above embodiment, operation 910 may also be omitted in the case where the overall coronal view of the breast can be acquired by one ultrasonic scanning.

In operation 920, the plurality of local coronal views each show a part of the breast. For example, a local coronal view acquired by performing ultrasonic scanning on an area containing the triangular marker and the circular marker shown in FIG. 5 illustrates a lower left area of the breast, a local coronal view acquired by performing ultrasonic scanning on an area containing the triangular marker and the square marker shown in FIG. 5 illustrates an upper left area of the breast, a local coronal view acquired by performing ultrasonic scanning on an area containing the square marker and a prismatic marker shown in FIG. 5 illustrates an upper right area of the breast, and a local coronal view acquired by performing ultrasonic scanning on an area containing the prismatic marker and the circular marker shown in FIG. 5 illustrates a lower right area of the breast. Thus, based on the embodiment of the present application, an overall coronal view of the breast can be acquired by splicing (registering) the four local coronal views.

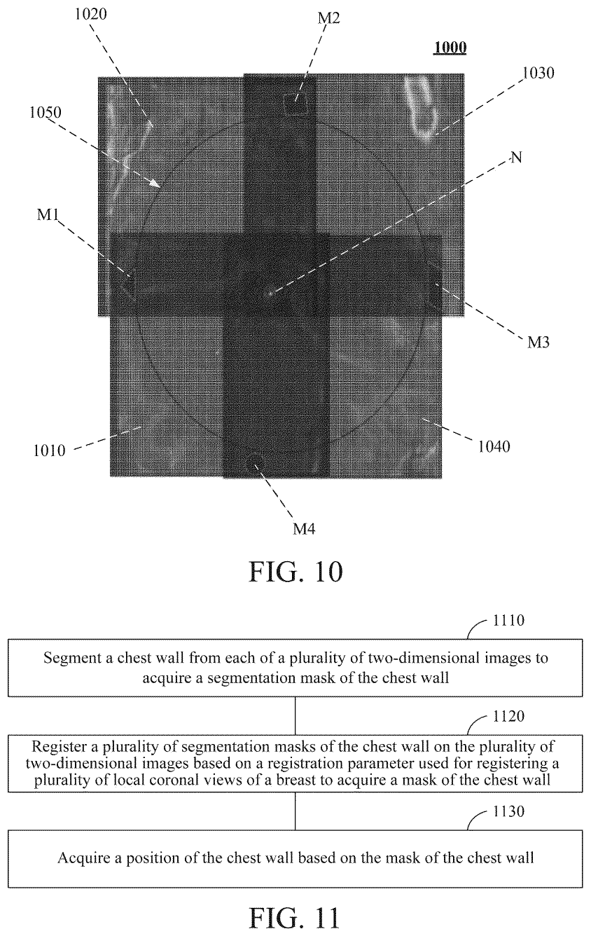

As shown in FIG. 10, positions of a nipple N on four local coronal views 1010 to 1040 are aligned, a triangle marker M1 on the local coronal view 1010 and a triangle marker M1 on the local coronal view 1020 are aligned, a square marker M2 on the local coronal view 1020 and a square marker M2 on the local coronal view 1030 are aligned, a prismatic marker M3 on the local coronal view 1030 and a prismatic marker M3 on the local coronal view 1040 are aligned, and a circular marker M4 on the local coronal view 1040 and a circular marker M4 on the local coronal view 1010 are aligned. Thus, an overall coronal view 1000 of the breast is acquired.

The above registering (splicing) method is only an example. In the case where the number of markers is changed and/or the shape of the markers are the same or similar, another registering (splicing) method may alternatively be used. Reference may be made to the related technology, which is not repeated herein. In addition, operation 920 may also be omitted in the case where the overall coronal view of the breast can be acquired by one ultrasonic scanning.

In operation 930, when the overall coronal view of the breast is acquired, since the markers are attached to the outer edge of the breast, a breast boundary can be determined based on the positions of the markers. For example, curve fitting is performed on the four markers shown in FIG. 5, and the four markers are connected to acquire the breast boundary. A connecting line 1050 shown in FIG. 10 is labeled as the breast boundary.

In operation 940, a volume of the breast can be acquired once the breast boundary and a distance from the skin to a chest wall within the boundary are known. The specific calculation method is not limited in the present application, and reference may be made to the related technology.

In the above embodiment, a position of the chest wall may be determined in a method shown in FIG. 11. As shown in FIG. 11, the method includes:
1110: segmenting a chest wall from each of a plurality of two-dimensional images to acquire a segmentation mask of the chest wall;
1120: registering a plurality of segmentation masks of the chest wall on the plurality of two-dimensional images based on a registration parameter used for registering a plurality of local coronal views of a breast to acquire a mask of the chest wall; and
1130: acquiring a position of the chest wall based on the mask of the chest wall.

It should be noted that FIG. 11 merely schematically illustrates the method for determining the position of the chest wall in the embodiment of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 11.

In operation 1110, the segmentation method is similar to operation 610 shown in FIG. 6 and is not repeated herein.

In operation 1120, a plurality of segmentation masks of the chest wall on the plurality of two-dimensional images are registered based on a registration parameter, such as a same registering transformation matrix, used for registering a plurality of local coronal views to acquire a mask of the chest wall. Further, a position of the chest wall may be acquired based on the mask of the chest wall.

In the above embodiment, when the position of the chest wall is determined, the distance from the skin to the chest wall can be acquired, and then the volume of the breast can be determined based on the breast boundary.

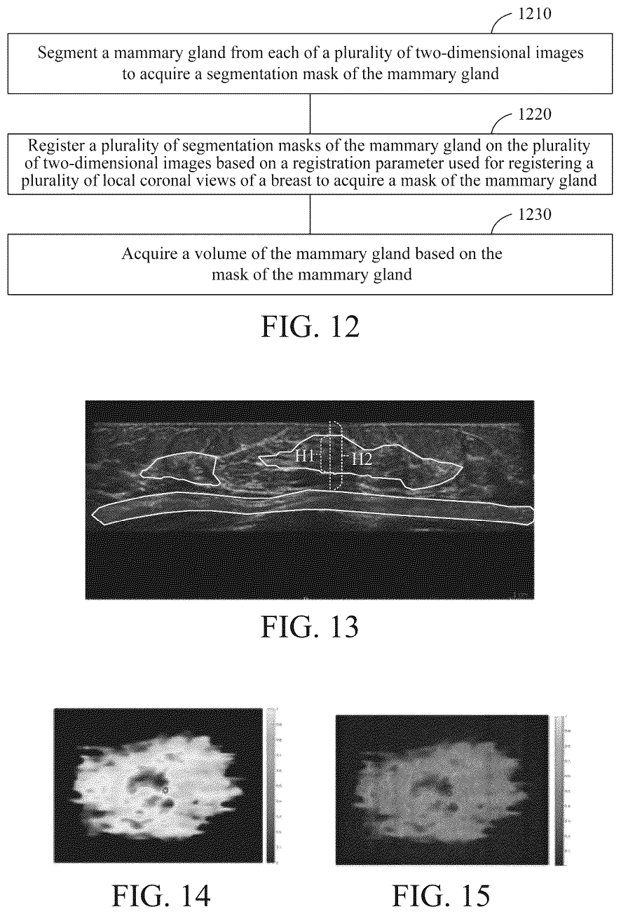

In operation 630, the determination of a volume of the mammary gland based on the mammary gland may be implemented, for example, in the method shown in FIG. 12. As shown in FIG. 12, the method includes:
1210: segmenting a mammary gland from each of a plurality of two-dimensional images to acquire a segmentation mask of the mammary gland;
1220: registering a plurality of segmentation masks of the mammary gland on the plurality of two-dimensional images based on a registration parameter used for registering a plurality of local coronal views of a breast to acquire a mask of the mammary gland; and
1230: acquiring a volume of the mammary gland based on the mask of the mammary gland.

It should be noted that FIG. 12 merely schematically illustrates the method for determining the volume of the mammary gland based on the mammary gland according to an embodiment of the present application, but the present application is not limited thereto. For example, the order of execution between operations may be appropriately adjusted. In addition, some other operations may be added or some operations may be omitted. Those skilled in the art may make appropriate variations according to the above content, rather than being limited to the above disclosure of FIG. 12.

In operation 1210, the segmentation method is similar to operation 610 shown in FIG. 6 and is not repeated herein.

In operation 1220, a plurality of segmentation masks of the mammary gland on the plurality of two-dimensional images are registered based on a registration parameter, such as a same registering transformation matrix, used for registering a plurality of local coronal views to acquire a mask of the mammary gland. Further, a volume of the mammary gland may be acquired from calculation based on the mask of the mammary gland, and the specific calculation method is not limited in the present application.

In the above embodiment, when the volume of the mammary gland and the volume of the breast are determined, a density of the breast may be acquired by dividing the volume of the mammary gland by the volume of the breast.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

In some other embodiments, as shown in FIG. 4, the method may further include:
440: calculating a breast density map within an area of the breast based on a thickness of the mammary gland and a distance from the skin to a chest wall; and
450: visualizing and displaying the breast density map.

In the above embodiment, the thickness of the mammary gland may be determined based on, for example, the ultrasonic image. For example, the thickness of the mammary gland may be acquired from calculation using the mask of the mammary gland. The specific calculation method is not limited in the present application. The mask of the mammary gland may be acquired in the method shown in FIG. 12, which is not repeated herein.

In the above embodiment, the position of the chest wall may be determined based on the ultrasonic image. For example, the position of the chest wall may be acquired in the method shown in FIG. 11, which is not repeated herein. The distance from the skin to the chest wall may be acquired based on the position of the chest wall.

FIG. 13 is a schematic diagram of calculation of a breast density map. As shown in FIG. 13, the mask of the breast is acquired in the method shown in FIG. 12. A thickness H1 of the breast may be acquired from calculation based on the mask of the breast. The position of the chest wall is acquired in the method shown in FIG. 11. A distance H2 from the skin to the chest wall may be acquired based on the position of the chest wall. In an embodiment of the present application, for each point within the breast boundary, the density of the breast is calculated, namely, H1/H2, and thus a breast density map within the range of the breast boundary is obtained. FIG. 14 is a schematic diagram of an example of the breast density map. As shown in FIG. 14, different densities may be represented by different colors or different depths of a color, and the present application is not limited thereto. The breast density map may also be represented in other manners. Based on the breast density map, a doctor or an ultrasonic scanning operator can visually detect a lesion, providing convenience for the operator.

In the above embodiment, optionally, the breast density map may also be overlaid and displayed on a coronal view of the breast. FIG. 15 is a schematic diagram of an example of overlaying and displaying the breast density map shown in FIG. 14 on a coronal view of the breast. As shown in FIG. 15, since the coronal view of the breast shows the full view of the breast, the detection of the lesion can be further guided by overlaying and displaying the breast density map.

According to the method in the embodiments of the present application, breast density is quantified by acquiring three-dimensional volume data of a breast, and detecting a glandular breast tissue and a chest wall tissue from the three-dimensional volume data. Optionally, distribution of the breast density may also be visualized and displayed, providing more information for clinical applications, such as lesion detection navigation.

The embodiments of the present application further provide an ultrasonic imaging system. The ultrasonic imaging system includes a scanning assembly for moving over the surface of a breast to acquire ultrasonic echo signals, and a processor. The specific implementation of the scanning assembly has been described above and will not be described again here.

In the above embodiment, the processor is configured to perform the method of the foregoing embodiments, and since the specific implementation of the method has been described in the foregoing embodiments, the contents of which are incorporated herein, no further description is provided herein.

The embodiments of the present application further provide a non-transitory computer-readable medium, having a computer program stored thereon, where the computer program has at least one code segment, and the at least one code segment is executable by a machine so that the machine performs steps of the method according to the foregoing embodiments. Since the specific implementation of the method has been described in the foregoing embodiments, the contents of which are incorporated herein, no further description is provided herein.

The above method of the present application may be implemented by hardware, or may be implemented by hardware in combination with software. The present application relates to such a computer-readable program that when executed by a logic component, the program enables the logic component to implement the constituent components described above, or enables the logic component to implement various methods or steps as described above. The present application further relates to a storage medium for storing the above program, such as a hard disk, a disk, an optical disk, a DVD, a flash memory.

The method described with reference to the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A method for quantifying breast density, comprising:
performing ultrasonic scanning on a breast to acquire an ultrasonic image of the breast;
determining a volume of a mammary gland and a volume of the breast based on the ultrasonic image; and
determining the breast density based on the volume of the mammary gland and the volume of the breast.

2. The method according to claim 1, wherein the performing ultrasonic scanning on a breast to acquire an ultrasonic image of the breast comprises:
performing, via a scanning assembly, ultrasonic scanning on the breast, of which an outer edge is attached with at least one acoustically opaque marker, to acquire the ultrasonic image,
wherein the scanning assembly comprises a frame, the frame accommodates a scanning probe and a driving device, and the driving device drives the scanning probe to move within the frame to perform the ultrasonic scanning.

3. The method according to claim 2, wherein the marker comprises a plurality of markers, and the plurality of markers are attached to the outer edge of the breast.

4. The method according to claim 1, wherein the ultrasonic image comprises a plurality of two-dimensional images.

5. The method according to claim 4, wherein the determination of a volume of a mammary gland and a volume of the breast based on the ultrasonic image comprises:
segmenting each of the plurality of two-dimensional images to acquire the mammary gland and a chest wall of the breast;
reconstructing each of the plurality of two-dimensional images to acquire a local coronal view of the breast; and
determining the volume of the mammary gland based on the mammary gland, and determining the volume of the breast based on the chest wall and the local coronal views.

6. The method according to claim 5, wherein the determination of the volume of the mammary gland based on the mammary gland comprises:
segmenting the mammary gland from each of the plurality of two-dimensional images to acquire a segmentation mask of the mammary gland;
registering the plurality of segmentation masks of the mammary gland on the plurality of two-dimensional images based on a registration parameter used for registering the plurality of local coronal views of the breast to acquire a mask of the mammary gland; and
acquiring the volume of the mammary gland based on the mask of the mammary gland.

7. The method according to claim 5, wherein the determination of the volume of the breast based on the chest wall and the local coronal views comprises:
detecting a nipple and markers on each of the plurality of local coronal views;
registering the plurality of local coronal views based on positions of the nipple and the markers on each of the local coronal views to acquire an overall coronal view of the breast;
determining a breast boundary based on the positions of the markers; and
acquiring the volume of the breast based on the breast boundary and a distance from the skin to the chest wall within the breast boundary.

8. The method according to claim 7, wherein the determination of the volume of the breast based on the chest wall and the plurality of local coronal views further comprises:
determining a position of the chest wall.

9. The method according to claim 8, wherein the determination of a position of the chest wall comprises:
segmenting the chest wall from each of the plurality of two-dimensional images to acquire a segmentation mask of the chest wall;
registering the plurality of segmentation masks of the chest wall on the plurality of two-dimensional images based on a registration parameter used for registering the plurality of local coronal views of the breast to acquire a mask of the chest wall; and
acquiring a position of the chest wall based on the mask of the chest wall.

10. The method according to claim 4, wherein the method further comprises:
calculating a breast density map within an area of the breast based on a thickness of the mammary gland and a distance from the skin to a chest wall; and
visualizing and displaying the breast density map.

11. The method according to claim 10, wherein the method further comprises:
overlaying and displaying the breast density map on a coronal view of the breast.

12. The method according to claim 10, wherein the method further comprises:
determining the thickness of the mammary gland and a position of the chest wall based on the ultrasonic image.

13. The method according to claim 12, wherein the determination of the thickness of the mammary gland based on the ultrasonic image comprises:
segmenting the mammary gland from each two-dimensional image to acquire a segmentation mask of the mammary gland;
registering the plurality of segmentation masks of the mammary gland on the plurality of two-dimensional images based on a registration parameter used for registering a plurality of local coronal views of the breast to acquire a mask of the mammary gland; and
acquiring the thickness of the mammary gland from calculation based on the mask of the mammary gland.

14. The method according to claim 12, wherein the determination of a position of the chest wall based on the ultrasonic image comprises:
segmenting the chest wall from each two-dimensional image to acquire a segmentation mask of the chest wall;
registering the plurality of segmentation masks on the plurality of two-dimensional images based on a registration parameter used for registering a plurality of local coronal views of the breast to acquire a mask of the chest wall; and
acquiring a position of the chest wall based on the mask of the chest wall.

15. An ultrasonic imaging system, comprising:
a scanning assembly moving over the surface of a breast to acquire ultrasonic echo signals;
a memory storing instructions; and
a processor configured to execute the instructions to:
perform ultrasonic scanning on a breast to acquire an ultrasonic image of the breast;
determine a volume of a mammary gland and a volume of the breast based on the ultrasonic image; and
determine the breast density based on the volume of the mammary gland and the volume of the breast.

16. The system according to claim 15, wherein the performing of ultrasonic scanning on a breast to acquire an ultrasonic image of the breast comprises:
performing, via the scanning assembly, ultrasonic scanning on the breast, of which an outer edge is attached with at least one acoustically opaque marker, to acquire the ultrasonic image,
wherein the scanning assembly comprises a frame, the frame accommodates a scanning probe and a driving device, and the driving device drives the scanning probe to move within the frame to perform the ultrasonic scanning.

17. The system according to claim 16, wherein the marker comprises a plurality of markers, and the plurality of markers are attached to the outer edge of the breast.

18. The system according to claim 15, wherein the ultrasonic image comprises a plurality of two-dimensional images.

19. The system according to claim 18, wherein the determination of a volume of a mammary gland and a volume of the breast based on the ultrasonic image comprises:
segmenting each of the plurality of two-dimensional images to acquire the mammary gland and a chest wall of the breast;
reconstructing each of the plurality of two-dimensional images to acquire a local coronal view of the breast; and
determining the volume of the mammary gland based on the mammary gland, and determining the volume of the breast based on the chest wall and the local coronal views.

20. A non-transitory computer-readable medium, having a computer program stored thereon, wherein the computer program has at least one code segment, and the at least one code segment is executable by a machine so that the machine performs steps of:
performing ultrasonic scanning on a breast to acquire an ultrasonic image of the breast;
determining a volume of a mammary gland and a volume of the breast based on the ultrasonic image; and
determining the breast density based on the volume of the mammary gland and the volume of the breast.
